(19) United States
(12) Patent Application Publication
(10) Pub. No.: US 2010/0137403 A1
(43) Pub. Date: Jun. 3, 2010

(54) METHOD FOR ENHANCING COGNITION OR INHIBITING COGNITIVE DECLINE

(76) Inventors: Scott Malstrom, Reading, MA (US); John J. Byrnes, Ashland, MA (US); Michelle Wolfe, Tyngsboro, MA (US)

Correspondence Address: HARNESS, DICKEY, & PIERCE, P.L.C, 7700 Bonhomme, Suite 400, ST. LOUIS, MO, 63105, US

(21) Appl. No.: 12/499,910

(22) Filed: Jul. 9, 2009

Related U.S. Application Data
(60) Provisional application No. 61/079,543, filed on Jul. 10, 2008.

(51) Int. Cl. A61K 31/385; A61P 25/00

(52) U.S. Cl. 514/436

(57) ABSTRACT

A method for enhancing cognition or inhibiting cognitive decline in a subject comprises selecting a Ca²⁺ channel blocker that is effective, when administered intravenously to an animal in a nontoxic amount, to increase NF-κB expression in the brain of the animal; and administering the selected Ca²⁺ channel blocker to the subject, via a systemic route that affords an adequate therapeutic window for cognition-enhancing or cognitive decline-inhibiting effectiveness of the selected Ca²⁺ channel blocker, in an amount within the therapeutic window. The selected Ca²⁺ channel blocker can be, for example, tiapamil or a pharmaceutically acceptable salt or prodrug thereof.

This application claims the benefit of U.S. provisional application Ser. No. 61/079,543 filed on Jul. 10, 2008, the disclosure of which is incorporated by reference herein in its entirety.

### FIELD OF THE INVENTION

The present invention relates to methods for enhancing cognition and/or for inhibiting decline of cognitive function in a subject in need thereof. More particularly, the invention relates to such methods comprising administering a pharmacotherapeutic agent.

### BACKGROUND

Nuclear factor κB (NF-κB) activation has been implicated as a mediator of certain neuronal functions. See, e.g., Meffert et al. (2003) Nature Neurosci. 6:1072-1078. Agents that modulate NF-κB transcription or activation in the brain are therefore of potential interest for treatment of central nervous system (CNS) disorders. An interplay exists between intracellular calcium (Ca²⁺) ions and NF-κB in neurons, increases in NF-κB activation being associated with increased intracellular Ca²⁺ concentration and with excitatory Ca²⁺-dependent neurotransmission. It has been reported that such NF-κB activation is inhibited by Ca²⁺ chelators, glutamate receptor antagonists, tetrodotoxin and L-type Ca²⁺ channel blockers. Id.

L-type Ca²⁺ channel blockers are widely used as antihypertensive agents. They include phenylalkylamines such as verapamil, benzothiazepines such as diltiazem and dihydropyridines such as nifedipine, nimodipine and amlodipine, among others. Chronic hypertension is well known to adversely affect cognitive function (see, e.g., Knopman et al. (2001) Neurology 56:42-48), and antihypertensive drugs including L-type Ca²⁺ channel blockers have been reported to bring cognitive as well as cardiovascular benefits.

For example, Murray et al. (2002) Arch. Intern. Med. 162:2090-2096 reported a survey of an older adult population of African Americans, in which it was found that antihypertensive medications reduced the odds of incident cognitive impairment by 38%. In the case of Ca²⁺ channel blockers specifically, odds of incident cognitive impairment were reduced less dramatically (14%) than in the case, for example, of angiotensin-converting enzyme (ACE) inhibitors (36%), antiadrenergics (73%) or diuretics (20%).

Maxwell et al. (1999) Can. Med. Assoc. J. 161(5):501-506 reported a five-year study of older Canadians in which odds of significant decline in cognitive performance were found to be substantially greater in subjects using Ca⁺ channel blockers than other antihypertensives.

Ban et al. (1990) Prog. Neuropsychopharmacol. Biol. Psychiatry 14:525-551 reported that in age-related cognitive decline (both Alzheimer's disease and vascular dementia), nimodipine was superior to placebo.

Tollefson (1991) Biol. Psychiatry 27:1133-1142 reported that in a series of 227 primary degenerative dementia patients, those treated with nimodipine showed less progression of the illness over 12 months than those treated with placebo.

In a review article Bojarski et al. (2007) Neurochemistry International 52:621-633 concluded that therapeutic strategies that aim to correct calcium dysregulation are likely to slow the progress of Alzheimer's disease.

Freir et al. (2003) J. Neurophysiol. 89:3061-3069 reported that verapamil attenuated β-amyloid-induced depression of long-term potentiation in a particular brain region (a correlate of memory function) in rats, and proposed that verapamil could be useful in treatment of cognitive deficits associated with Alzheimer's disease.

Walker et al. (1985) Neurology 25(Suppl. 1):177 reported no effect of diltiazem in Huntington's disease.

Hollister & Garza Trevino (1999) Can. J. Psychiatry 44:658-664, in a review of use of Ca²⁺ channel blockers in psychiatric practice, concluded with respect to nifedipine that “the poor therapeutic compared with possible side effects” would militate against use in psychiatric disorders; and with respect to verapamil that not enough evidence was available to accept verapamil as an effective therapeutic agent for psychiatric disorders, other than possibly for mania. However, they did remark that the studies of Ban et al. (1990), supra and Tollefson (1991), supra on age-related dementias were “somewhat encouraging, considering the dire implications of these disorders.”

Bojanova et al. (1997) Meth. Find. Exp. Clin. Pharmacol. 19(2):93-97 reported that verapamil at 10 mg/kg completely abolished amnesia induced by electroconvulsive shock or by clonidine, and proposed that verapamil might be useful for treatment of cognitive disorders.

Popović et al. (1997) Int. J. Neurosci. 90:87-97 tested verapamil in a two-way active avoidance learning study in nucleus basalis magnocellularis lesioned rats, a model for Alzheimer's disease. They reported that verapamil at 2.5 and 5 mg/kg improved both acquisition and performance aspects of active avoidance, but that lower (1 mg/kg) and higher (10 mg/kg) doses were ineffective.

Quartermain et al. (2001) Neurobiol. Learning Memory 75:77-90 reported that of six Ca²⁺ channel blockers tested in young adult mice only one, verapamil (the only phenylalkylamine included in the study) failed to facilitate retention of passive avoidance learning in a dose-dependent manner. Verapamil reportedly showed enhancement effects at three doses in linear maze retention, but even at the most effective retention dose failed to enhance acquisition.

Palmer et al. (1990) Br. J. Clin. Pharmacol. 30:365-370 reported that in a long-term clinical trial without placebo control, nifedipine was associated with a 31% deterioration, and verapamil with a 22% improvement, in cognitive function. They acknowledged that the apparent positive effect of verapamil could have been simply due to inclusion in the trial (i.e., an uncontrolled placebo effect).

Cárdenas et al. (1998) Eur. Neuropsychopharmacal. 8:187-189 reported that nimodipine and verapamil had no effect on verbal learning in a placebo-controlled clinical trial.

Liesi et al. (1997) J. Neurosci. Res. 48:571-579 reported that verapamil could restore genetically-inhibited neurite extension in mouse cerebellar neurons, and could also be neuroprotective in normal neurons exposed to high concentrations of ethanol. They suggest evaluation of verapamil for treatment of alcohol-induced brain disorders and neurodegenerative diseases.

Moser et al. (2004) Stroke 35:e369-e372 reported that vasodilatation in response to administration of verapamil was correlated with neuropsychological performance in elderly patients with atherosclerosis but in whom dementia had not been diagnosed.

Wauquier et al. (1985) Jap. J. Pharmacol. 38:1-7 compared nine Ca²⁺ channel blockers for efficacy on aspects of ischemic disease. They reported that the phenylalkylamine Ca²⁺ channel blockers verapamil, D-600 (gallopamil) and tiapamil, together with the benzothiazepine Ca²⁺ channel blocker diltiazem, were ineffective, attributing this possibly to poor brain penetration.

Kortekaas et al. (2005) Ann. Neurol. 57:176-179 reported ability of verapamil to cross the blood-brain barrier, but noted that this ability was significantly greater in Parkinson's disease patients than in control subjects.

U.S. Patent Application Publication No. 2004/0254176 of Grigorieff et al. proposes treatment of any of a wide range of diseases by administering a combination of an ACE inhibitor, a Ca²⁺ channel blocker and a diuretic. Diseases said to be treatable by the method include cognitive dysfunction such as Alzheimer's disease. Among Ca²⁺ channel blockers said to be useful (although not preferred) are verapamil, gallopamil and tiapamil.

U.S. Patent Application Publication No. 2005/0222137 of Shetty & Webb proposes treatment of any of a wide range of diseases by administering a combination of an angiotensin receptor blocker, a Ca²⁺ channel blacker and a diuretic. Diseases said to be treatable by the method include cognitive dysfunction such as Alzheimer's disease. Among Ca²⁺ channel blockers said to be useful are verapamil, gallopamil and tiapamil.

U.S. Patent Application Publication No. 2005/0153953 of Trippodi-Murphy et al. proposes treatment of memory and/or cognitive impairment by administering a combination of an L-type Ca²⁺ channel blocker, more particularly a dihydropyridine, and a cholinesterase inhibitor.

U.S. Patent Application Publication No. 2007/0142475 of Selhier et al. proposes treatment of any of a wide range of diseases by administering a specified type of renin inhibitor. Diseases said to be treatable by the method include cognitive disorders. Optionally the renin inhibitor can be administered in combination with a second agent, for example a Ca²⁺ channel blocker such as verapamil, gallopamil or tiapamil.

International Patent Publication No. WO 2007/003941 of Cambridge University Technical Services Ltd. proposes treatment of diseases that are ameliorated by induction of autophagy by administering a calpain inhibitor. Such diseases are said to include neurodegenerative diseases such as Huntington's disease, Parkinson's disease, Pick's disease, Alzheimer's disease, Lewy body dementia, variant Creutzfeld-Jacob disease (CJD), etc. Among a large number of compounds said to function as calpain inhibitors are L-type Ca²⁺ channel blockers such as verapamil, gallopamil and thiapamil (tiapamil).

Despite a great number of publications, some more encouraging than others as illustrated above, relating to possible use of Ca²⁺ channel blockers in treatment of cognitive disorders, there remains a need in the art for a method for selecting a particular Ca²⁺ channel blacker having superior potential in this regard. This need is particularly acute given the adverse side-effect profile and narrow therapeutic window of many Ca²⁺ channel blockers.

### SUMMARY OF THE INVENTION

It has now been surprisingly discovered that at least some Ca²⁺ channel blockers, when administered systemically to an animal in vivo, cause increased expression of NF-κB in certain areas of the brain, specifically in sub-anatomical regions involved in sensory perception, filtering, emotion, learning and memory. It has also been surprisingly observed that no significant increase in NF-κB expression occurs in other parts of the body.

Follow-up testing in an animal model of cognitive, more specifically memory, performance has confirmed that such Ca²⁺ channel blockers can be effective in enhancing cognition.

At doses effective to provide the observed increase in NF-κB activation, not all Ca²⁺ channel blockers are free of adverse side effects. The L-type Ca²⁺ channel blocker tiapamil, for example, has a greater margin of selectivity for the desired NF-κB expression increase in the brain than other phenylalkylamines such as verapamil and gallopamil.

Accordingly there is now provided a method for enhancing cognition or inhibiting cognitive decline in a subject, comprising:
selecting a Ca²⁺ channel blocker that is effective, when administered intravenously to an animal in a nontoxic amount, to increase NF-κB expression in the brain of the animal; and
administering the selected Ca²⁺ channel blocker to the subject, via a systemic route that affords an adequate therapeutic window for cognition-enhancing or cognitive decline-inhibiting effectiveness of the selected Ca²⁺ channel blocker, in an amount within the therapeutic window.

There is further provided a method for enhancing cognition or inhibiting cognitive decline in a subject, comprising administering tiapamil or a pharmaceutically acceptable salt or prodrug thereof to the subject, via a systemic route that affords an adequate therapeutic window for cognition-enhancing or cognitive decline-inhibiting effectiveness of tiapamil, in an amount within the therapeutic window.

There is still further provided a method for enhancing cognition or inhibiting cognitive decline in a subject, comprising systemically administering (a) a Ca²⁺ channel blocker to the subject in a cognition-enhancing or cognitive decline-inhibiting effective amount, and (b) an agent that counteracts non-brain-specific adverse effects of the Ca²⁺ channel blocker.

There is still further provided a method for enhancing cognition or inhibiting cognitive decline in a normotensive subject, comprising administering a Ca²⁺ channel blocker to the subject, via a systemic route that affords an adequate therapeutic window for cognition-enhancing or cognitive decline-inhibiting effectiveness of the Ca²⁺ channel blacker, in an amount within the therapeutic window.

### BRIEF DESCRIPTION OF THE DRAWINGS

FIG. 1 is a graphical representation of results of a study described in Example 1 showing that tiapamil activates NF-κB signaling in the brain.

FIG. 2 is a graphical representation of results of a study described in Example 2 showing that tiapamil activates NF-κB signaling in several sub-anatomical regions in the brain.

FIG. 3 is a graphical representation of results of a study described in Example 3 showing that the phenylalkylamine Ca²⁺ channel blockers tiapamil, verapamil and gallopamil all activate NF-κB signaling in the brain.

FIG. 4 is a graphical representation of results of a study described in Example 4 showing that tiapamil, administered i.v., has a therapeutic window for activation of NF-κB signaling in the brain, but that i.v. doses of verapamil and gallopamil providing similar activation are lethal to mice.

FIG. 5 is a graphical representation of results of a study described in Example 5 showing that effects of tiapamil on NF-κB activation in the brain can be suppressed by sulfasalazine.

FIG. 6 is a graphical representation of results of a study described in Example 6 showing that tiapamil can induce short-term memory enhancement in healthy mice and reverse short-term memory loss in scopolamine-treated mice.

FIG. 7 is a graphical representation of results of a study described in Example 7 showing that tiapamil-induced short-term memory enhancement in mice requires NF-κB signaling.

FIG. 8 is a graphical representation of results of a study described in Example 8 showing effect of acute administration of tiapamil on long-term (novel object recognition) memory in healthy rats.

### DETAILED DESCRIPTION

Methods are provided herein for enhancing cognition or inhibiting cognitive decline in a subject. A “subject” herein can be of any animal species, more particularly any mammalian species including primates, farm and work animals such as horses, domestic pets such as dogs and cats, exotic animals including captive and zoo animals, laboratory animals such as rats, mice and other rodents, etc. Preferably the subject is a primate, more especially a human subject. Human subjects can be of either gender and of any age. A human subject who can benefit from practice of the present methods is typically one having a cognitive deficit or in a state of cognitive decline, which can be simply age-related or associated with a neurodegenerative condition such as any of those mentioned hereinbelow. A human subject is typically, but not necessarily, a patient under the care of a physician or clinician who can be a generalist or a specialist such as a neurologist or psychiatrist. A patient can be in the community or in a residential care facility.

The phrase “enhancing cognition” or “cognitive enhancement” herein means increasing the level of at least one aspect of cognitive performance over a baseline level prior to treatment according to a method as provided herein. For example, according to some embodiments of the present invention, cognitive enhancement is achieved in a subject having a cognitive deficit that is stable, i.e., not in continuing decline. According to other embodiments, the subject has a cognitive deficit that is ameliorating with time, for example during natural or medically assisted recovery from traumatic, tumor-related or ischemic brain injury. In such a subject, a method of the present invention can provide cognitive enhancement to a greater degree or in a shorter period of time than would occur otherwise. Cognitive enhancement can be, but is not necessarily, assessed by comparison with placebo treatment.

The phrase “inhibiting cognitive decline” or “cognitive decline inhibition” herein embraces any of slowing, retarding, delaying, reducing, arresting and reversing progress of decline in the level of at least one aspect of cognitive performance. In other words, cognitive decline inhibition is marked by the subject exhibiting a higher level of at least one aspect of cognitive performance than (s)he would have exhibited in absence of treatment according to a method as provided herein, but not necessarily a higher level than at baseline. Cognitive decline inhibition can be, but is not necessarily, assessed by comparison with placebo treatment.

Aspects of cognitive performance which can be improved, or decline in which can be slowed, retarded, delayed, reduced, arrested or reversed, include without limitation memory acquisition, memory retention, sensory perception, learning, verbal and numerical skills, social skills, communication skills, etc. A beneficial effect on at least one such aspect can represent successful treatment, but in many cases more than one aspect of cognitive performance exhibits a beneficial response.

A “cognitive deficit disorder” herein means any disorder in which the subject exhibits an abnormally low level of at least one aspect of cognitive performance, but in whom a neurodegenerative disease has not been or cannot be diagnosed. Cognitive deficit disorders treatable by methods provided herein include without limitation learning disorders, memory disorders, sensory perception disorders, attention deficit/hyperactivity disorder, cognitive deficits associated with autism or Asperger's syndrome, mild cognitive impairment, age-related cognitive decline, cognitive impairment associated with traumatic, tumor-related or ischemic brain injury (including acute cerebrovascular events such as stroke, hemorrhage, embolism, thrombosis or rupturing aneurysm), drug- or alcohol-related cognitive impairment, and the like.

In some embodiments, the subject exhibits cognitive decline that is associated with a neurodegenerative disease, whether diagnosed clinically or not. Neurodegenerative diseases in which cognitive decline can occur include without limitation vascular dementia, Alzheimer's disease (including early-onset and familial Alzheimer's disease), Pick's disease, Lewy body dementia, presenile dementia, CJD, variant CJD, Parkinson's disease, Huntington's disease, neurodegeneration in Down syndrome, HIV-related dementia, and the like.

Memory Pharmaceuticals Corp. recently reported a clinical trial of MEM 1003, a derivative of nimodipine, in patients with mild to moderate Alzheimer's disease. The primary endpoint, a 12-week mean change in the cognitive subscale of the Alzheimer's disease assessment scale (ADAS-cog), was not met. See news release dated Oct. 15, 2007 at phx.corporate-ir.net/phoenix.zhtml?c=175500&p=irol-newsArticle&t=Regular&id=1062734&, incorporated by reference herein without admission as to its status as prior art or otherwise with respect to the present invention.

International Patent Publication No. WO 2007/112288 of Mount Sinai School of Medicine proposes, inter alia, use of any of a miscellany of about 60 cardiovascular agents said to have potential for reducing β-amyloid plaque development, for treatment of Alzheimer's disease. One such agent listed is verapamil. This publication is incorporated by reference herein without admission as to its status as prior art or otherwise with respect to the present invention.

Some neurodegenerative diseases are characterized by deposition of protein aggregates in the brain, for example mutant hungtingtin protein in the case of Huntington's disease, and plaque-foiling β-amyloid protein in the case of Alzheimer's disease. Use of L-type Ca²⁺ channel blockers has been proposed for induction of autophagy through inhibition of calpain to reduce deposition of or clear such protein aggregates (see above-cited International Patent Publication No. WO 2007/003941).

In one embodiment of the present invention, the subject has a cognitive deficit or neurodegenerative disorder that is not ameliorated by induction of autophagy. Such a disorder is generally one not characterized by deposition of protein aggregates in the brain. In a particular embodiment, a method for enhancing cognition or inhibiting cognitive decline in a subject comprises systemically administering a therapeutically effective amount of tiapamil or a pharmaceutically acceptable salt or prodrug thereof to the subject, wherein the subject has a cognitive deficit disorder or neurodegenerative condition that is not ameliorated by induction of autophagy. Examples of neurodegenerative conditions that do not necessarily involve deposition of protein aggregates and are not ameliorated by autophagy include without limitation vascular dementia, presenile dementia, neurodegeneration in Down syndrome and HIV-related dementia.

It will be recognized that if a selected Ca²⁺ channel blocker has activity both as a calpain inhibitor and, in accordance with the present invention, as a brain-selective NF-κB activator, it is unlikely to show equal potency for both effects. In one embodiment, where the subject has a neurodegenerative disorder such as Alzheimer's disease or Huntington's disease that is ameliorated by induction of autophagy, the selected Ca²⁺ channel blocker is administered at an effective dose for increasing NF-κB expression in the brain that is lower (for example at least about 2× lower, or at least about 4× lower) than a minimum effective dose of the same Ca²⁺ channel blocker for induction of autophagy in the brain. In another embodiment, where again the subject has a neurodegenerative disorder such as Alzheimer's disease or Huntington's disease that is ameliorated by induction of autophagy, a minimum effective dose of the selected Ca²⁺ channel blocker for increasing NF-κB expression in the brain is at least about 2× higher (for example at least about 4× higher) than a minimum effective dose of the same Ca²⁺ channel blocker for induction of autophagy in the brain. According to this embodiment, the selected Ca²⁺ channel blocker is administered at a dose that is not lower than that minimum effective dose for increasing NF-κB expression in the brain.

The terms “disorder,” “disease” and “condition” are used interchangeably herein, unless the particular context demands that a distinction be drawn.

Unless the context demands otherwise, the terms “treat,” “treating” or “treatment” herein include preventive or prophylactic use of an agent in a subject at risk of, or having a prognosis including, cognitive deficit or decline, as well as use of such an agent in a subject already experiencing cognitive deficit or decline. Thus treatment includes (a) preventing cognitive deficit or decline from occurring in a subject that may be predisposed to a neurodegenerative disorder but in whom such a disorder has not yet been diagnosed, (b) inhibiting cognitive decline, and/or (c) enhancing cognition in a subject having a cognitive deficit. The terms “prevent,” “preventing,” “prevention” and “preventive” will be understood to have their normal meaning in the medical arts of reducing risk or future incidence or severity of a disorder, or of one or more symptoms thereof, as opposed to total elimination of future occurrence of the disorder or symptoms.

Cognitive performance can be measured according to any standardized scale known in the art appropriate to the particular aspect or aspects of cognitive performance which are to be enhanced or decline in which is to be inhibited. For example, the cognitive subscale of the Alzheimer's disease assessment scale (ADAS-cog) is useful in measuring levels of various aspects of cognitive performance in subjects having Alzheimer's disease and other dementias. Other suitable scales for measuring cognitive performance are known to those of skill in the art.

In one embodiment, a method of the invention comprises selecting a Ca²⁺ channel blocker that is effective, when administered intravenously to an animal in a nontoxic amount, to increase NF-κB expression in the brain of the animal.

The set of Ca²⁺ channel blockers from which selection is made can encompass all agents having Ca²⁺ channel blocking or antagonist activity, but L-type Ca²⁺ channel blockers, including without limitation phenylalkylamines, dihydropyridines and benzothiazepines, are generally preferred. In one embodiment, selection is made from L-type Ca²⁺ channel blockers of the phenylalkylamine class. Non-limiting examples of phenylalkylamines include anipamil; bepridil; devapamil (also known as 4-desmethoxyverapamil or D-888); fendiline; gallopamil (also known as methoxyverapamil or D-600); prenylamine; ronipamil; tiapamil (also known as thiapamil, dimeditiapramine or RO-11-1781) and derivatives thereof including RO-11-2933; verapamil and its metabolite noverapamil; and YS-035. Any of these can be used in its free base form or as a pharmaceutically acceptable salt. The hydrochloride salt is often the most convenient, but other salts can be substituted if desired, including without limitation hydrobromate, acetate, oxalate, malonate, succinate, maleate, fumarate, phthalate, terephthalate, ascorbate, glycolate, lactate, malate, tartrate, citrate, aspartate, glutamate, benzoate, mesylate and tosylate salts and the like. Ca²⁺ channel blockers that exhibit stereoisomerism can be used as single enantiomers or as any mixture of enantiomers, including racemic mixtures. Prodrugs of Ca²⁺ channel blockers can also be used.

In a particular embodiment the Ca²⁺ channel blocker selected is tiapamil (N-(3,4-dimethoxyphenethyl)-2-(3,4-dimethoxyphenyl)-N-methyl-m-dithian-2-propylamin-1,1,3,3-tetroxide), for example in the foul) of its hydrochloride (HCl) salt.

The Ca²⁺ channel blocker selected must meet the test criterion set forth above, namely that a nontoxic amount administered intravenously to an animal results in an increase in NF-κB expression in the brain of the animal.

The animal used to establish this criterion is preferably a laboratory animal, more preferably a rodent, illustratively a mouse. Any known technique can be used to measure the degree of NF-κB expression in the brain of the animal, including but not limited to the in vivo technique described in Example 1 hereof. In this technique, a test compound, in this case a Ca²⁺ channel blocker, is administered by intravenous (i.v.) injection to transgenic mice having a firefly luciferase reporter driven by one or more NF-κB response elements fused to the reporter (NF-κB::LUC mice). NF-κB expression, nuclear translocation and binding to the response elements in any anatomical or sub-anatomical region of a mouse results in activation of the luciferase reporter in that region. Activated luciferase, in presence of a luciferin substrate, emits light which can be detected and quantitated, and can be used in whole-body imaging to identify specific anatomical or sub-anatomical regions where the luciferase is activated as a result of NF-κB expression. Mice can be anesthetized and imaged at time intervals after administration of the test compound to reveal temporal as well as spatial distribution of NF-κB expression.

The test compound can be administered in different dosage amounts (usually expressed as mg/kg body weight) to establish one or more doses, or a range of doses, at which increased NF-κB expression in the brain is observed. Systemic health of the test animals is monitored for evidence of toxicity, especially lethality, at each dose. If at least one dose, or a range of doses, is identified that is nontoxic to the test animals but causes increased NF-κB expression in the brain, the test compound meets the present criterion and can be selected for administration according to the method of the present embodiment.

Although selection can be on the basis of increased NF-κB expression in the brain as a whole, in a preferred technique NF-κB expression is quantitated or imaged in one or more specific sub-anatomical regions of the brain known to be involved in cognitive processing. These regions include the olfactory bulb, dorsal hippocampus, cingulate cortex, caudate nucleus, thalamus, hypothalamus and cerebellar vermis. In particular embodiments, the criterion for selection comprises increased NF-κB expression in at least one, at least two, at least three, at least four, at least five, at least six, or all seven of these regions.

Not all Ca²⁺ channel blockers meet a criterion for selection as set forth above. As shown in Example 4, tiapamil administered i.v. at 10 mg/kg was nonlethal to mice and substantially increased NF-κB expression in the brain versus control. However, although verapamil and gallopamil also increased NF-κB expression in the brain, i.v. administration of these compounds resulted in deaths of mice even at the lower doses tested (2 mg verapamil, 1 mg gallopamil).

A Ca²⁺ channel blocker, selected based on the criterion discussed above, is administered to a subject in need of cognition enhancement or cognitive decline inhibition via a systemic route that affords an adequate therapeutic window for cognition-enhancing or cognitive decline-inhibiting effectiveness of the selected Ca²⁺ channel blocker, in an amount within the therapeutic window.

The term “therapeutic window” compares, for a particular compound administered by a particular systemic route, a minimum dose effective to provide an acceptable degree of cognition enhancement or inhibition of cognitive decline, with a maximum dose that can be tolerated by the subject without unacceptable adverse side-effects. It can be expressed as a dose range from minimum effective to maximum tolerable dose (e.g., 100-400 mg/day), or as a ratio of maximum tolerable to minimum effective dose (e.g., 4:1). If the maximum tolerable dose of a compound is lower than the minimum effective dose (i.e., corresponding to a ratio<1:1), there is no therapeutic window for the compound. The selection step described above can be expected to eliminate from consideration many compounds lacking a therapeutic window, but is not an absolute guarantee that a selected compound will have a therapeutic window in clinical practice. If the compound has a therapeutic window (i.e., corresponding to a ratio>1:1), it should be administered at a dose within that window. Preferably the compound and route of administration are selected to provide a therapeutic window corresponding to a ratio of maximum tolerable to minimum effective dose of at least about 2:1, more preferably at least about 3:1, most preferably at least about 4:1.

A “systemic route” of administration can be any route that delivers the selected Ca²⁺ channel blocker to the bloodstream of the subject, whence it is carried throughout the body. Systemic routes include without limitation parenteral (including intravenous, subcutaneous and intradermal), transdermal, transmucosal (including rectal, intraoral and intranasal) and peroral (p.o.) routes. The term “oral” or “orally” applied to a route of administration herein will be understood to mean peroral, i.e., involving delivery to the gastrointestinal tract via the mouth, as opposed to intraoral, i.e., involving delivery across oral mucosa as in sublingual or buccal administration.

The route of administration chosen can affect the therapeutic window of a selected Ca²⁺ channel blocker. For example, intravenous administration can be expected to provide a relatively narrow therapeutic window, as it provides an immediate pulse of the Ca²⁺ channel blocker to the cardiovascular system where the greatest potential for adverse side-effects is typically present. This can be moderated to some extent by employing a slow infusion rather than bolus injection mode of i.v. administration.

Most Ca²⁺ channel blockers are orally bioavailable, and oral administration is generally the most convenient route, especially for non-hospitalized patients. Oral administration also typically affords a wider therapeutic window than i.v. administration.

It is believed, without being bound by theory, that the cognitive benefits of methods of the present invention are mediated at least in part by increased NF-κB expression, selectively in the brain and more particularly in regions of the brain involved in cognitive processing, as discussed above. It is further believed, again without being bound by theory, that the effect on NF-κB expression in the brain is an indication of at least some transport of the selected Ca²⁺ channel blocker across the blood-brain barrier.

The discovery of the brain-selective NF-κB expression effect of Ca²⁺ channel blockers, in particular L-type Ca²⁺ channel blockers such as phenylalkylamines including tiapamil, gallopamil and verapamil, has led to an important insight, namely that the Ca²⁺ channel blocker selected for use according to the present method be one having as strong as possible a cognition-enhancing or cognitive decline-inhibiting effect while having a relatively weak cardiovascular (e.g., systemic antihypertensive or vasodilatory) effect. Whether or not transport across the blood-brain barrier is involved in providing the cognitive effects noted herein, it is likely that the amount or concentration of the Ca²⁺ channel blocker in the cardiovascular system is much greater than that in the CNS, hence the importance of a relatively weak cardiovascular effect (to minimize systemic side-effects) accompanying as strong as possible a cognitive effect. Much of the focus in the art relating to cognitive benefits of Ca²⁺ channel blockers has been on overcoming cognitive deficits or decline associated with hypertension, for example through vasodilatory effects of Ca²⁺ channel blockers in the vasculature of the brain. In keeping with this focus, Ca²⁺ channel blockers with particularly potent cardiovascular effects, such as verapamil and dihydropyridines such as nifedipine and nimodipine, have hitherto been favored for study in neurodegenerative diseases such as Alzheimer's disease having a major cognitive component. The findings of the present inventors enable the focus to be shifted to Ca²⁺ channel blockers with relatively weak cardiovascular effects, illustratively tiapamil.

Thus the present invention provides a new approach to treatment of cognitive deficit or neurodegenerative disorders that are not merely a consequence of hypertensive disease. Accordingly, in one embodiment the subject is normotensive, i.e., having systolic and diastolic blood pressures in a healthy range in the absence of medical intervention. A “healthy range” herein is about 80/40 mmHg to about 140/90 mmHg (in each case expressed as systolic/diastolic blood pressure), more particularly about 90/50 mmHg to about 120/80 mmHg. By selecting a Ca²⁺ channel blocker such as tiapamil that is a relatively weak antihypertensive, the concomitant risk of inducing hypotension (a systolic and/or diastolic blood pressure below the healthy range) in an otherwise normotensive subject is lower than in the case of a more potent antihypertensive such as, for example, verapamil.

However, in any therapeutic method of the invention, it will generally be desirable to monitor the subject's blood pressure, at least for the first few weeks or months of therapy. Such monitoring can be done by the subject himself/herself, or by a health-care professional such as a nurse or physician, for example in a clinic or medical office. If hypotension occurs, or blood pressure falls toward a hypotensive state (for example a reduction in systolic and/or diastolic blood pressure of more than about 20 mmHg), consideration can be given to reducing the dose of the Ca²⁺ channel blocker and/or co-administering an antihypotensive drug in an amount effective to bring blood pressure back within a desired range. Antihypotensives include vasoconstrictors such as antihistamines and amphetamines.

In one embodiment, a method for enhancing cognition or inhibiting cognitive decline in a subject comprises administering a Ca²⁺ channel blocker, e.g., tiapamil or a pharmaceutically acceptable salt or prodrug thereof, to the subject, via a systemic route that affords an adequate therapeutic window for cognition-enhancing or cognitive decline-inhibiting effectiveness of the Ca²⁺ channel blocker, in an amount within the therapeutic window, wherein the subject is normotensive and has a cognitive deficit disorder or neurodegenerative condition that is other than a protein aggregation disorder and/or that is not ameliorated by induction of autophagy.

In another embodiment, a method for enhancing cognition or inhibiting cognitive decline in a subject comprises systemically administering (a) a Ca²⁺ channel blocker, e.g., tiapamil or a pharmaceutically acceptable salt or prodrug thereof, to the subject in a cognition-enhancing or cognitive decline-inhibiting effective amount, and (b) an agent that counteracts a non-brain-specific adverse side-effect of the Ca²⁺ channel blocker. The non-brain-specific side-effect can be, for example, a cardiovascular side-effect such as hypotension, in which case the agent that counteracts the side-effect can be an antihypotensive, for example a vasoconstrictor as mentioned above.

A daily (per diem) dose of a Ca²⁺ blocker useful herein will depend on the particular Ca²⁺ channel blocker selected and can be titrated depending on the particular subject's response and on occurrence of any adverse side-effects. Illustratively for tiapamil, a suitable daily dose is likely to be found in a range of about 1 to about 50 mg/kg body weight, for example about 2 to about 25 mg/kg body weight. For an adult human subject having a body weight of about 40 to about 100 kg, a suitable daily dose of tiapamil can be, for example, about 50 to about 2000 mg, more typically about 100 to about 1500 mg or about 200 to about 1200 mg. Illustrative daily doses include, without limitation, about 100, about 150, about 200, about 250, about 300, about 400, about 500, about 600, about 750, about 1000, about 1200 or about 1500 mg. Where tiapamil is administered in a form of a salt or prodrug thereof, doses of the salt or prodrug equivalent to the above doses of tiapamil free base can be used. One of ordinary skill in the art can select suitable daily doses of Ca²⁺ channel blockers other than tiapamil without undue experimentation based on disclosure herein.

The above doses are given on a per diem basis but should not be interpreted as necessarily being administered on a once daily frequency. Indeed the compound, or salt or prodrug thereof, can be administered at any suitable frequency, for example as determined conventionally by a physician taking into account a number of factors, but typically about four times a day, three times a day, twice a day, once a day, every second day, twice a week, once a week, twice a month or once a month. The compound, or salt or prodrug thereof, can alternatively be administered more or less continuously, for example by parenteral infusion in a hospital setting. In some situations a single dose may be administered, but more typically administration is according to a regimen involving repeated dosage over a treatment period. In such a regimen the daily dose and/or frequency of administration can, if desired, be varied over the course of the treatment period, for example introducing the subject to the compound at a relatively low dose and then increasing the dose in one or more steps until a full dose is reached.

The treatment period is generally as long as is needed to achieve a desired outcome, for example a particular degree of improvement or attainment of a goal on a cognitive performance scale such as ADAS-cog. In some situations it will be found useful to administer the Ca²⁺ channel blocker intermittently, for example for treatment periods of days, weeks or months separated by non-treatment periods. In other situations it will be found useful to administer the Ca²⁺ channel blocker continuously and more or less indefinitely, especially where the subject has a progressive neurodegenerative disease.

While it can be possible to administer the Ca²⁺ channel blocker, as free base, salt or prodrug, unformulated as active pharmaceutical ingredient (API) alone, it will generally be found preferable to administer the API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below.

USIP, ed. (2005) Remington: The Science and Practice of Pharmacy, 21st ed., Lippincott, Williams & Wilkins.

Allen et al. (2004) Ansel's Pharmaceutical Dosage Forms and Drug Delivery Systems, 8th ed., Lippincott, Williams & Wilkins

Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present invention include, without limitation, solutions, suspensions, powders, granules, tablets, capsules, pills, lozenges, chews, creams, ointments, gels, liposomal preparations, nanoparticulate preparations, injectable preparations, enemas, suppositories, inhalable powders, sprayable liquids, aerosols, patches, depots and implants.

Illustratively, in a liquid formulation suitable, for example, for parenteral, intranasal or oral delivery, the API can be present in solution or suspension, or in some other form of dispersion, in a liquid medium that comprises a diluent such as water. Additional excipients that can be present in such a formulation include a tonicifying agent, a buffer (e.g., a tris, phosphate, imidazole or bicarbonate buffer), a dispersing or suspending agent and/or a preservative. Such a formulation can contain micro- or nanoparticulates, micelles and/or liposomes. A parenteral formulation can be prepared in dry reconstitutable form, requiring addition of a liquid carrier such as water or saline prior to administration by injection.

For rectal delivery, the API can be present in dispersed form in a suitable liquid (e.g., as an enema), semi-solid (e.g., as a cream or ointment) or solid (e.g., as a suppository) medium. The medium can be hydrophilic or lipophilic.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC or hypromellose), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Crosearmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

A pharmaceutical composition useful herein typically contains the compound or salt or prodrug thereof in an amount of about 1% to about 99%, more typically about 5% to about 90% or about 10% to about 60%, by weight of the composition. A unit dosage form such as a tablet or capsule can conveniently contain an amount of the compound providing a single dose, although where the dose required is large it may be necessary or desirable to administer a plurality of dosage forms as a single dose. Illustratively for tiapamil, a unit dosage form can comprise the compound in an amount of about 50 to about 2000 mg, for example about 100 to about 1500 mg or about 200 to about 1200 mg. Illustrative daily doses include, without limitation, about 100, about 150, about 200, about 250, about 300, about 400, about 500, about 600, about 750, about 1000, about 1200 or about 1500 mg. Where tiapamil is present in the unit dosage form as a salt or prodrug, amounts of the salt or prodrug equivalent to the above doses of tiapamil free base can be present in the unit dosage form. One of ordinary skill in the art can select suitable amounts of Ca²⁺ channel blockers other than tiapamil for preparation of unit dosage forms without undue experimentation based on disclosure herein.

In some cases, the selected Ca²⁺ channel blocker will be one of a plurality of active agents administered for cognitive enhancement or inhibition of cognitive decline. In some cases, the selected Ca²⁺ channel blocker will be administered for cognitive enhancement or inhibition of cognitive decline concomitantly with one or more additional active agents for treatment of an associated condition. An “associated condition” herein can be one that is secondary to cognitive deficit or decline, for example depression or other mood disorders. Alternatively or in addition, an “associated condition” herein can be one to which cognitive deficit or decline is secondary, for example a neurodegenerative disorder or traumatic or ischemic brain injury. In some cases, the selected Ca²⁺ channel blocker will be administered for cognitive enhancement or inhibition of cognitive decline concomitantly with one or more additional agents for reducing or correcting adverse side-effects of the Ca²⁺ channel blocker such as hypotension or other cardiovascular side-effects. Each of the above situations is referred to herein as combination therapy.

The two or more active agents administered in combination can be formulated in one pharmaceutical preparation (single dosage form) for administration to the subject at the same time, or in two or more distinct preparations (separate dosage forms) for administration to the subject at the same or different times, e.g., sequentially. The two distinct preparations can be formulated for administration by the same route or by different routes.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in a first container, a first agent that comprises a Ca²⁺ channel blocker, e.g., tiapamil or a salt or prodrug thereof, and, in a second container, a second agent as indicated above. In another example, the first and second agents are separately packaged and available for sale independently of one another, but are co-marketed or co-promoted for use according to the invention. The separate dosage forms may also be presented to a subject separately and independently, for use according to the invention.

Depending on the dosage forms, which may be identical or different, e.g., fast release dosage forms, controlled release dosage forms or depot forms, the first and second agents may be administered on the same or on different schedules, for example on a daily, weekly or monthly basis.

A therapeutic combination comprising a Ca²⁺ channel blocker, for example tiapamil or a pharmaceutically acceptable salt or prodrug thereof, and an antihypotensive agent, for example a vasoconstrictor such as an antihistamine or an amphetamine, is a further embodiment of the present invention. In a particular embodiment, the Ca²⁺ channel blocker is present in an amount effective to enhance cognition or inhibit cognitive decline in a subject having need thereof, and the antihypotensive agent is present in an amount effective to counteract a hypotensive side-effect of the Ca²⁺ channel blocker. The combination can comprise separate dosage forms of the Ca²⁺ channel blocker and the antihypotensive agent, for example separately packaged or co-packaged, or can have both the Ca²⁺ channel blocker and the antihypotensive agent co-formulated in the same dosage form.

### Examples

### Example 1

### Tiapamil Dose-Dependently Increases NF-κB Activation in the Brain

In vivo bio-photonic imaging was used to investigate temporal and spatial modulation of physiological processes following acute tiapamil administration to mice. For these studies we employed an NF-κB luciferase transgenic line that was constructed using three NF-κB response elements fused to a firefly luciferase reporter gene. The effect of tiapamil on NF-κB activation was monitored as modulation of detectable luciferase reporter activity, a surrogate for NF-κB activation and nuclear translocation with subsequent binding to the response elements of the reporter. Luciferase activity was detected by its ability to cleave luciferin, a luciferase substrate. Light emitted was detected and analyzed using a highly sensitive CCD imaging system.

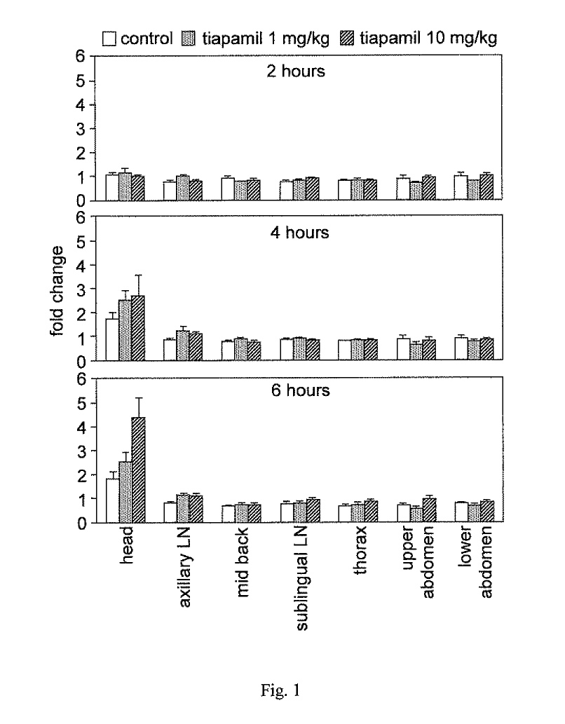

For the present study, female NF-κB transgenic mice (5 per treatment) received tiapamil i.v. at 1 or 10 mg/kg in a saline vehicle, or vehicle control. At 2, 4 and 6 hours after administration, the animals were anesthetized and imaged. Visual and quantitative analysis was then performed based on counting photons of light emitted from specific anatomical regions. Imaging revealed a dose-dependent increase in NF-κB expression in the head of animals at 4 and 6 hours following tiapamil treatment. Tiapamil had no effect on NF-κB activation in any of the peripheral anatomical regions evaluated. The data are depicted quantitatively in FIG. 1.

### Example 2

### Tiapamil Activates NF-κB in Several Sub-Anatomical Regions in the Brain

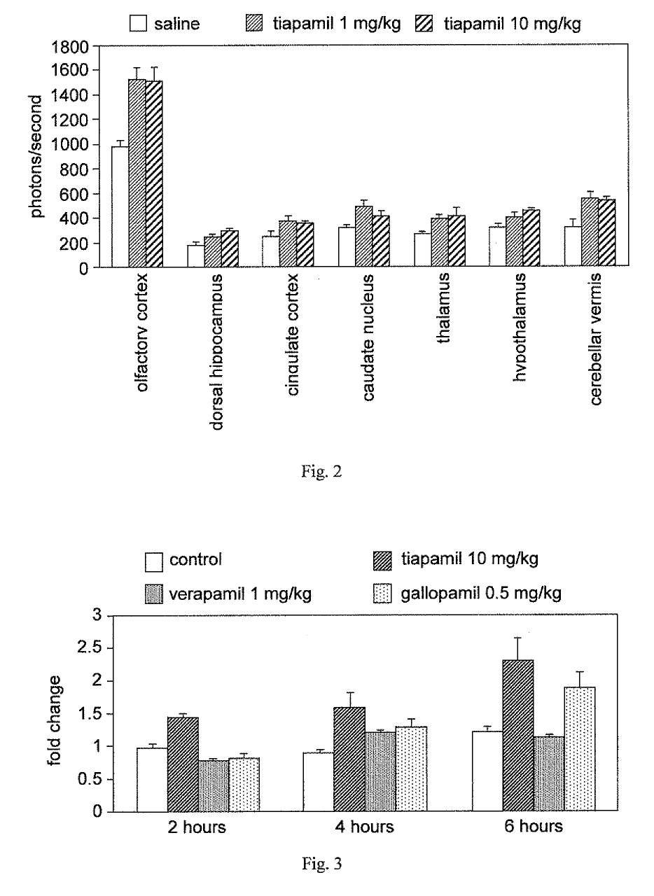

Evaluation of tiapamil in the NF-κB transgenic mouse line was repeated essentially as described in Example 1 and whole-body images were collected at the 6-hour time point. Brains were rapidly removed, chilled in ice-cold saline and sliced into 1 mm coronal sections. The sections were then placed onto glass slides, covered with luciferin solution (Luciferase Assay System substrate, Promega) and subsequently imaged in an IVIS 200 imaging system (Caliper Corp.) to evaluate effect of tiapamil on regional distribution of luciferase activity in the brain. As shown in FIG. 2, tiapamil had a dramatic effect on luciferase expression in all regions of the brain evaluated. Interestingly, the magnitude of the effect was similar for both 1 and 10 mg/kg doses of the drug.

### Example 3

### Activation of NF-κB in the Brain is a Class Effect of Phenylalkylamines

A study was performed to compare effects of tiapamil and two other phenylalkylamine Ca²⁺ channel blockers, verapamil and gallopamil on inducing NF-κB activation in mouse brain. Drug doses were chosen to be reflective of the potency of each of the drugs in blocking L-type calcium channel function. Evaluation of tiapamil (10 mg/kg), verapamil (1 mg/kg) and gallopamil (0.5 mg/kg) in the NF-κB transgenic mouse line was repeated essentially as described for tiapamil in Example 1, by i.v. administration, and dorsal brain images were obtained at the 2-, 4- and 6-hour time points. As shown in FIG. 3, tiapamil exhibited a robust effect on measurable NF-κB activation at the three time points evaluated. In contrast, verapamil exhibited only a marginal effect on detectable NF-κB activation at all time points measured. Gallopamil also exhibited a marginal effect at the 2- and 4-hour time points and a more dramatic effect at the 6-hour time point.

### Example 4

### Tiapamil has a Therapeutic Window for Activation of NF-κB in the Brain

As described in Example 3, it was found that tiapamil (10 mg/kg) exhibited greater NF-κB activation effect than either verapamil (1 mg/kg) or gallapomil (0.5 mg/kg). The doses administered for these studies were selected to reflect the previously characterized potency of each of the drugs on blocking L-type Ca²⁺ channel function. However, in order to more fully explore a possible dose dependent activity of phenylalkylamine calcium channel blockers to activate NF-κB in the mouse brain, additional studies were performed utilizing increased doses of both verapamil and gallopamil. A study was performed essentially as described for tiapamil in Example 1 using the following chosen doses: tiapamil (10 mg/kg), verapamil (2 and 10 mg/kg) and gallopamil (1 and 10 mg/kg). Unexpectedly, it was found that increasing the dose for both verapamil and gallopamil resulted in a corresponding increase in death in the study mice (Table 1).

TABLE 1
Lethality of phenylalkylamine Ca²⁺ channel blockers in mice

| Treatment | Deaths (n = 5) |
|---|---|
| Control | 0 |
| tiapamil 10 mg/kg | 0 |
| verapamil 2 mg/kg | 3 |
| verapamil 10 mg/kg | 5 |
| gallopamil 1 mg/kg | 2 |
| gallopamil 10 mg/kg | 4 |

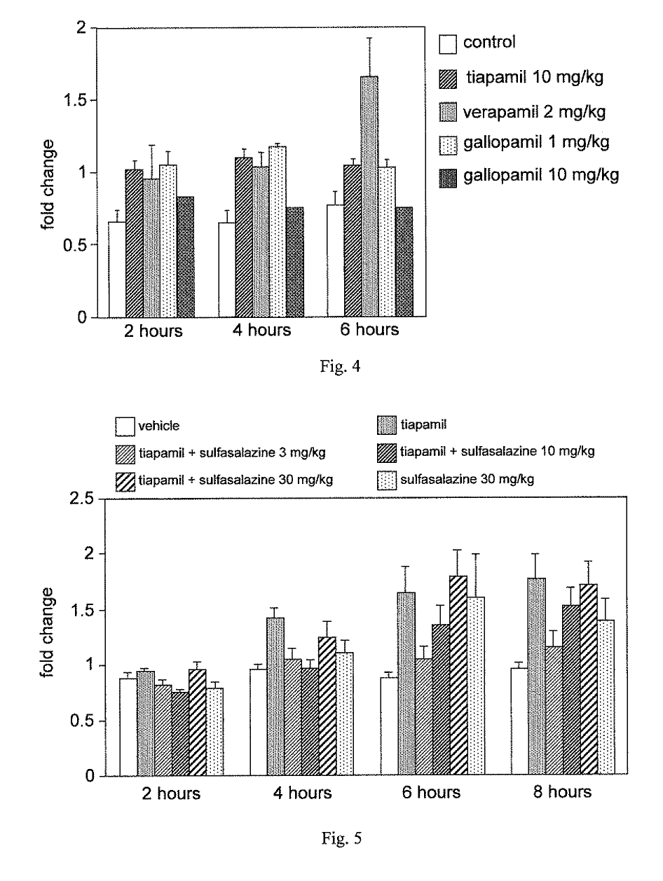

For those mice that did not perish following drug administration, standard dorsal brain images were obtained at the 2-, 4- and 6-hour time points and images were processed as described above. As shown in FIG. 4, tiapamil once again exhibited a robust effect on NF-κB activation in the brain. Verapamil (2 mg/kg; 2 surviving animals) and gallopamil (1 mg/kg; 3 surviving animals) also induced NF-κB activation. In contrast, high-dose gallopamil (10 mg/kg; 1 surviving animal) exhibited minor to no change in NF-κB activation. Imaging studies evaluating high-dose verapamil (10 mg/kg) could not be performed as no animals survived acute drug administration. In summation, these findings indicate that tiapamil is the only phenylalkylamine of those tested capable of robustly activating NF-κB in the brain without elevated risk for inducing an acute lethal response following drug administration. This therapeutic window is an important differentiating element for tiapamil among members of the phenylalkylamine class of L-type Ca²⁺ channel blockers.

### Example 5

### Tiapamil-Induced NF-κB Signaling Activation is Suppressed by Sulfasalazine

A study was performed to address whether tiapamil-induced NF-κB activation in the CNS could repressed by co-administration of sulfasalazine, a known inhibitor of the signal transduction cascade controlling NE-κB activation. For this study, transgenic mice (as in the above examples) were pre-imaged prior to intraperitoneal (i.p.) co-administration of tiapamil and sulfasalazine. Images were acquired at 2, 4, 6 and 8 hours after administration and quantitative analysis of light emission from the head region (dorsal view) was performed. As shown in FIG. 5, tiapamil enhanced NF-κB signaling in the brain and this effect was blunted by co-administration of 3 mg/kg of sulfasalazine. The 3 mg/kg dose of sulfasalazine was chosen for subsequent studies to test whether tiapamil activation of NF-κB was causally related to improved memory function (as described below in Example 7). Administration of high-dose sulfasalazine or co-administration of tiapamil and high-dose sulfasalazine (further detailed in FIG. 5) resulted in increased amount of measureable NF-κB activity. These findings are inconsistent with the expected dampening effect of sulfasalazine on NF-κB activation and imply an unanticipated and presumably indirect effect of sulfasalazine on regulating NF-κB function.

### Example 6

### Tiapamil Enhances Short-Term Memory

NF-κB activation in the brain has been implicated in acquisition of short-term and long-term memory. See, for example, the publications individually cited below and incorporated herein by reference.

Guerrini et al. (1995) Proc. Natl. Acad. Sci. USA 92:9618-9622.

Meffert et al. (2003) Nature Neurosci. 6:1072-1078.

Cruise et al. (2000) Neuroreport 11:395-398.

Mattson et al. (2004) Trends Neurosci. 27:589-594.

Based on the findings reported above that tiapamil increases NF-κB selectively in the brain, studies were undertaken to test the hypothesis that tiapamil treatment can enhance short-term memory in a rodent model. The spontaneous alternation task (SAT) test is used to assess spatial “working” or short-term memory in rats and mice (see Pizzi & Spano (2006) Eur. J. Pharmacol. 545:22-28). The task is based on the innate, unlearned response of rodents to explore the novel environment of a previously unexplored arm of a T- or Y-maze. For example, if a healthy rat or mouse enters the left arm of a T-maze, the probability of entering the right arm on the next trial (i.e., alternation) is over 70%. That is, a rodent typically remembers which arm it has previously entered. Spontaneous alternation is impaired by the cholinergic antagonist scopolamine, and enhanced by the cholinesterase inhibitor donepezil. The SAT test is commonly used to study cognitive phenotypes in knockout and transgenic animals and as a pharmacological screen of cognitive enhancers. Thus, the objective of this study was to test the cognitive enhancing properties of tiapamil using the SAT test of short-term memory in healthy male mice.

Adult male C57BL/6 mice (Charles River Laboratory, Kingston, N.Y.) 6-8 weeks of age and weighing 19-21 g were housed five to a cage in a Thoren System rack. Each cage contained Alpha-dri bedding and environmental enrichment devices. Food (Rodent Chow 5001) and filtered tap water were provided ad libitum throughout the study. The animal housing facility was maintained on a 12 hour light/dark cycle. Temperature and relative humidity were recorded daily and were maintained in a range of 16-27° C. and 30-70% respectively. Animals were identified with tail numbers. All procedures conformed to normal standards for care and use of laboratory animals.

Following acclimation to the facility, animals were randomly assigned to receive either tiapamil (30 mg/kg, i.p.) or its 5% dimethyl sulfoxide (DMSO) vehicle 8 hours prior to the SAT. The rationale for this pre-treatment interval is based on in vivo imaging findings suggesting that tiapamil robustly induces brain NF-κB 8 hours post-treatment. Thirty minutes prior to the SAT, animals from each group above received either scopolamine (1 mg/kg, i.p.) or its 0.9% NaCl vehicle. Drug and vehicle solutions were delivered in a volume of 10 ml/kg. Animals from each of the 4 groups (10 animals per group) were then individually placed in the start box of a T-maze (start box: 48 cm long×14 cm wide×22 cm high; arms 50 cm long×14 cm wide×22 cm high) and were allowed to freely explore all arms. Once an animal entered one of the arms, it was confined for a total of 1 minute. The animal was then removed from the arm and placed back into the start box for the next trial. Each animal was given 8 trials. The number of alternation events was calculated and the percent alternation was used as a measure of short-term memory.

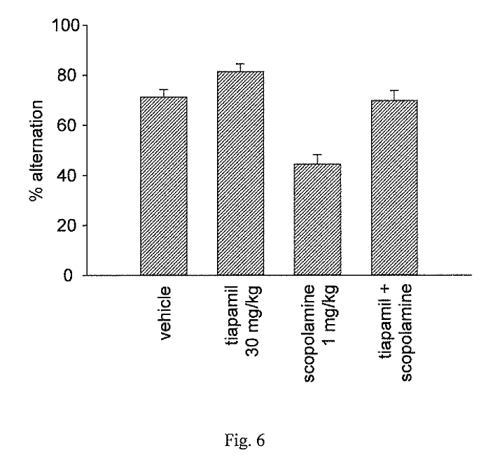

FIG. 6 depicts the results of 2 independent studies. Vehicle-treated control mice displayed approximately 70% alternation and this response was markedly impaired in scopolamine-treated animals, findings which are consistent with reports in the literature. Tiapamil both increased the number of alternation events when administered alone, and prevented scopolamine-induced impairment. These findings indicated that tiapamil, when administered 8 hours prior to testing, enhances short-term memory in healthy mice, suggesting its utility as a cognitive enhancer. Moreover, the finding that tiapamil reverses memory deficits in scopolamine-treated mice suggests that this compound may be beneficial in treatment of memory impairment induced by cholinergic deficits (e.g., Alzheimer's disease).

### Example 7

### Tiapamil-Induced Short-Term Memory Enhancement Requires NF-κB Signaling

Given the finding above that tiapamil enhances short-term memory in healthy mice, a study was undertaken to test the hypothesis that this effect requires intact NF-κB signaling. For this study, ability of the NF-κB inhibitor sulfasalazine to prevent tiapamil-induced improvement in the SAT was investigated.

Adult male C57BL/6 mice (Charles River Laboratory, Kingston, N.Y.) 6-8 weeks of age and weighing 19-21 g were housed five to a cage in a Thoren System rack. Each cage contained Alpha-dri bedding and environmental enrichment devices. Food (Rodent Chow 5001) and filtered tap water were provided ad libitum throughout the study. The animal housing facility was maintained on a 12 hour light/dark cycle. Temperature and relative humidity were recorded daily and were maintained in a range of 16-27° C. and 30-70% respectively. Animals were identified with tail numbers. All procedures conformed to normal standards for care and use of laboratory animals. General procedures including T-maze dimensions were similar to those described in Example 6 above.

Following acclimation to the facility, animals were randomly assigned to receive either Tiapamil (30 mg/kg, i.p.) or its 5% DMSO vehicle 8 hours prior to the SAT. Thirty minutes prior to the SAT, animals from each group above received either scopolamine (1 mg/kg, i.p.) or its 0.9% NaCl vehicle. To specifically test the hypothesis under consideration, an additional group was co-administered tiapamil and sulfasalazine (3 mg/kg, i.p.) 8 hours prior to the SAT. Drug and vehicle solutions were delivered in a volume of 10 ml/kg. Animals from each of the 5 groups (10 animals per group) were then individually placed in the start box of a T-maze and were allowed to freely explore all arms. Once an animal entered one of the arms, it was confined for a total of 1 minute. The animal was then removed from the arm and placed back into the start box for the next trial. Each animal was given 8 trials. The number of alternation events was calculated and the percent alternation was used as a measure of short-term memory.

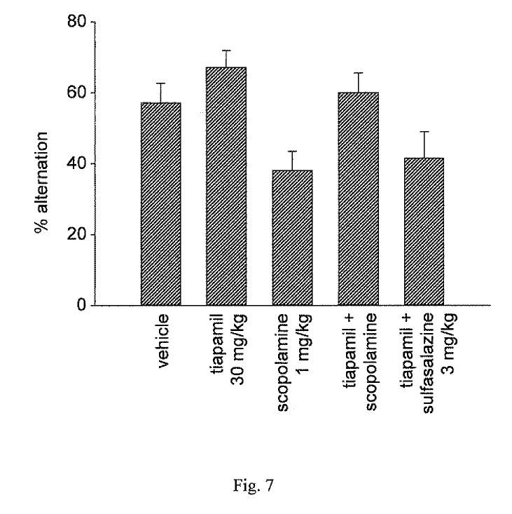

The results depicted in FIG. 7 support findings of the previous study (Example 6) that tiapamil both enhances short-term memory in healthy mice, and reverses memory deficits in scopolamine-treated animals. Co-administration of tiapamil and sulfasalazine to healthy (Le., untreated with scopolamine) mice resulted in a loss of tiapamil's memory-enhancing effects. The results indicate that the cognitive effects of tiapamil require or are mediated by intact NF-κB signaling.

### Example 8

### Tiapamil Enhances Novel Object Recognition Memory

The novel object recognition (NOR) paradigm is a rodent model of recognition learning memory retrieval and takes advantage of the spontaneous behavior of rodents to investigate a novel object by comparison with a familiar object (Ennaceur & Delacour (1988) Behav Brain Res. 31:47-59). NOR has been employed extensively to indicate potential cognition-enhancing properties of test compounds. The paradigm does not involve appetitive or aversive reinforcement such as food reward or noxious stimulus, thus providing one less confounding variable when translating from preclinical recognition memory tests to analogous testing conducted in human clinical trials.

The objective of this study was to test cognition-enhancing properties of tiapamil using the NOR test of long-term memory in healthy male rats. Adult male Sprague-Dawley rats were used in this study. Animals were placed in the experimental rooms at postnatal day 80 and assigned unique identification numbers (tail marked). Pairs were housed in polycarbonate cages with filter tops and acclimated for 7 days prior to commencing any studies. Animals were maintained in a 12 hour light/dark cycle with room temperature maintained at 22±2° C. with relative humidity maintained at approximately 50%. Food and water were provided ad libitum. All animals were examined, handled, and weighed for two days prior to initiation of the study to assure adequate health and suitability and to minimize non-specific stress associated with manipulation. Each animal was randomly assigned across treatment groups (8 animals per group) and balanced by cage numbers. The NOR experiment was performed during the animal's light cycle phase.

Tiapamil (10 or 30 mg/kg) was dissolved in saline and administered i.p. 1 or 8 hours prior to training. Galantamine (3 mg/kg), used as a positive control in this study, was dissolved in 0.9% saline and administered i.p. 1 hour prior to training. Galantamine is a competitive, reversible inhibitor of acetylcholinesterase and is used clinically to treat mild to moderate vascular dementia and Alzheimer's disease. The rats were assessed for cognitive ability in a test apparatus comprising an open-field arena placed in a sound-attenuated room under dimmed lighting. Each rat was tested separately and care was taken to remove any olfactory/taste cues by cleaning the arena and test objects with alcohol between trials and rats. All tests were video-scored blind. On day 1 (habituation), rats were allowed to explore an empty test area for 10 minutes each. On day 2, each rat was placed facing the same direction at the same position in the arena, and allowed to explore two identical objects for 10 minutes. This 10-minute training period, T1, was recorded for subsequent analysis if necessary. The rat was returned to its home cage between tests. After 24 hours, each rat was placed again in the test arena for 10 minutes (T2) in presence of a copy of the familiar object and a novel object, and the time spent exploring both objects was recorded. The presentation position of the objects (left/right) was randomized between rats to prevent bias from place preference. The NOR index was measured as the ratio of time spent exploring the novel object over total time spent exploring both objects (familiar+novel), during retention session T2.

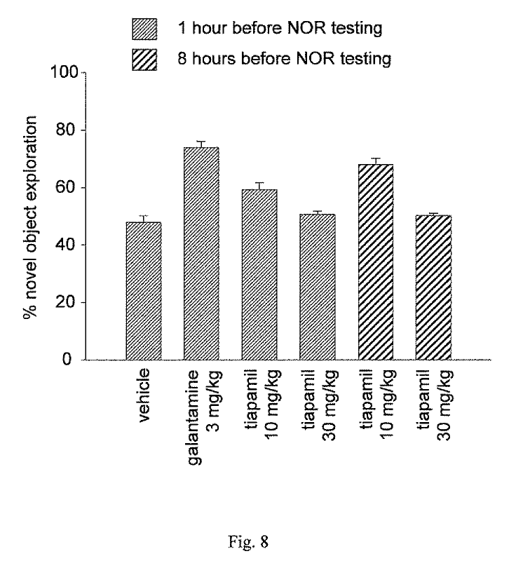

Effects of galantamine and tiapamil on the NOR index 24 hours after training are shown in FIG. 8. Post-hoc Student's t-test further confirmed that compared to vehicle, galantamine 3 mg/kg, 1 hour pre-NOR testing, significantly increased NOR index. Tiapamil at 10 mg/kg, whether administered 1 hour or 8 hours pre-NOR testing, also significantly increased NOR index. Tiapamil at 30 mg/kg was not effective to increase NOR index in this study. The effect of tiapamil on NOR was more robust when administered 8 hours than 1 hour pre-NOR testing. This finding is consistent with the temporal effects of tiapamil on effects of activation of NF-κB in the brain seen, for example, in Example 1 (FIG. 1). Tiapamil at 30 mg/kg did not effectively increase NOR index in this study. In summary, these findings suggest that Tiapamil may have clinical usefulness for memory enhancement and disorders of cognitive impairment.

Additional background information relating to methods used in the above Examples can be found in the publications individually cited below and incorporated by reference herein.

Meffert & Baltimore (2005) Trends Neurosci. 28:37-43.

Merlo et al. (2005) Learning & Memory 12:23-29.

Denis-Donini et al. (2008) J. Neurosci. 28:3911-3919.

Yao et al. (2007) Eur. J. Pharmacol. 574:20-28.

Boersma & Meffert (2008) Science Signaling 1(6):pe7.

Hughes (2004) Neurosci. Biobehay. Rev. 28:497-505.

Robe et al. (2004) Clin. Cancer Res. 10:5595-5603.

D'Acquisto & Ianaro (2006) Curr. Opin. Pharmacol. 6:387-392.

Medhurst et al. (2007) J. Pharmacol. Exp. Ther. 321:1032.

All patents and publications cited herein are incorporated by reference into this application in their entirety.

The words “comprise”, “comprises”, and “comprising” are to be interpreted inclusively rather than exclusively.

What is claimed is:

1. A method for enhancing cognition or inhibiting cognitive decline in a subject, comprising:
selecting a Ca²⁺ channel blocker that is effective, when administered intravenously to an animal in a nontoxic amount, to increase NF-κB expression in the brain of the animal; and
administering the selected Ca²⁺ channel blocker to the subject, via a systemic route that affords an adequate therapeutic window for cognition-enhancing or cognitive decline-inhibiting effectiveness of the selected Ca²⁺ channel blocker, in an amount within the therapeutic window.

2. The method of claim 1, wherein the Ca²⁺ channel blocker is tiapamil or a pharmaceutically acceptable salt or prodrug thereof.

3. The method of claim 2, wherein the subject is an adult human and the tiapamil or salt or prodrug thereof is administered in a daily tiapamil dose of about 50 to about 2000 mg.

4. The method of claim 1, wherein the subject has a cognitive deficit disorder and the administration of the selected Ca²⁺ channel blacker results in cognitive enhancement.

5. The method of claim 1, wherein the subject has cognitive decline associated with a neurodegenerative condition, and following administration of the selected Ca²⁺ channel blocker, the cognitive decline is inhibited.

6. The method of claim 1, wherein the subject has a cognitive deficit disorder or neurodegenerative condition other than a protein aggregation disorder.

7. The method of claim 1, wherein the systemic route of administration is peroral.

8. A method for enhancing cognition or inhibiting cognitive decline in a subject having a cognitive deficit disorder or neurodegenerative condition that is not ameliorated by induction of autophagy, the method comprising systemically administering a therapeutically effective amount of tiapamil or a pharmaceutically acceptable salt or prodrug thereof to the subject.

9. The method of claim 8, wherein the subject is an adult human and the tiapamil or salt or prodrug thereof is administered in a daily tiapamil dose of about 50 to about 2000 mg.

10. The method of claim 8, wherein the subject has a cognitive deficit disorder and the administration of the tiapamil or salt or prodrug thereof results in cognitive enhancement.

11. The method of claim 10, wherein the cognitive deficit disorder is selected from the group consisting of learning disorders, memory disorders, sensory perception disorders, attention deficit/hyperactivity disorder, cognitive deficits associated with autism and Asperger's syndrome, mild cognitive impairment, age-related cognitive decline, cognitive impairments associated with traumatic, tumor-related and ischemic brain injuries, cognitive impairments associated with stroke, hemorrhage, embolism, thrombosis and rupturing aneurysm, drug- and alcohol-related cognitive impairments, and combinations thereof.

12. The method of claim 8, wherein the subject has cognitive decline associated with a neurodegenerative condition that is not ameliorated by induction of autophagy, and following administration of the tiapamil or salt or prodrug thereof, the cognitive decline is inhibited.

13. The method of claim 12, wherein the neurodegenerative condition is selected from the group consisting of vascular dementia, presenile dementia, neurodegeneration in Down syndrome, HIV-related dementia, and combinations thereof.

14. The method of claim 8, wherein the systemic route of administration is peroral.

15. A method for enhancing cognition or inhibiting cognitive decline in a normotensive subject having a cognitive deficit disorder or neurodegenerative condition that is not ameliorated by induction of autophagy, the method comprising administering a Ca²⁺ channel blocker to the subject, via a systemic route that affords an adequate therapeutic window for cognition-enhancing or cognitive decline-inhibiting effectiveness of the Ca²⁺ channel blocker, in an amount within the therapeutic window.

16. The method of claim 15, wherein the Ca²⁺ channel blocker is tiapamil or a pharmaceutically acceptable salt or prodrug thereof.

17. The method of claim 16, wherein the subject is an adult human and the tiapamil or salt or prodrug thereof is administered in a daily tiapamil dose of about 50 to about 2000 mg.

18. The method of claim 15, wherein the subject has a cognitive deficit disorder and the administration of the Ca²⁺ channel blocker results in cognitive enhancement.

19. The method of claim 15, wherein the subject has cognitive decline associated with a neurodegenerative condition that is not ameliorated by induction of autophagy, and following administration of the Ca²⁺ channel blacker, the cognitive decline is inhibited.

20. The method of claim 15, wherein the systemic route of administration is peroral.

21. A method for enhancing cognition or inhibiting cognitive decline in a subject, comprising systemically administering (a) a Ca²⁺ channel blocker to the subject in a cognition-enhancing or cognitive decline-inhibiting effective amount, and (b) an agent that counteracts a non-brain-specific adverse side-effect of the Ca²⁺ channel blocker.

22. The method of claim 21, wherein the Ca²⁺ channel blocker is tiapamil or a pharmaceutically acceptable salt or prodrug thereof.

23. The method of claim 22, wherein the subject is an adult human and the tiapamil or salt or prodrug thereof is administered in a daily tiapamil dose of about 50 to about 2000 mg.

24. The method of claim 21, wherein the non-brain-specific adverse side-effect is a cardiovascular side-effect.

25. The method of claim 24, wherein the cardiovascular side-effect is hypotension.

26. The method of claim 25, wherein the agent that counteracts the hypotension is a vasoconstrictor.

27. A therapeutic combination comprising a Ca²⁺ channel blocker and an antihypotensive agent.

28. The combination of claim 27, wherein the Ca²⁺ channel blocker is tiapamil or a pharmaceutically acceptable salt or prodrug thereof.

29. The combination of claim 27, wherein the antihypotensive agent is a vasoconstrictor.

30. The combination of claim 27, wherein the Ca²⁺ channel blacker is present in an amount effective to enhance cognition or inhibit cognitive decline in a subject having need thereof, and the antihypotensive agent is present in an amount effective to counteract a hypotensive side-effect of the Ca²⁺ channel blocker.
